(19) United States
(12) Patent Application Publication
(10) Pub. No.: US 2003/0068653 A1
(43) Pub. Date: Apr. 10, 2003

(54) Determining existence of complications in pregnancies by measuring levels of bioactive lipids

(76) Inventor: Jeff A. Parrott, Irvine, CA (US)

Correspondence Address: ORRICK, HERRINGTON & SUTCLIFFE, LLP, 4 PARK PLAZA, SUITE 1600, IRVINE, CA, 92614-2558, US

(21) Appl. No.: 10/267,556

(22) Filed: Oct. 8, 2002

Related U.S. Application Data
(63) Continuation-in-part of application No. 09/585,138, filed on Jun. 1, 2000, now Pat. No. 6,461,830.

(51) Int. Cl. G01N 33/53; G01N 33/92

(52) U.S. Cl. 435/7.1; 436/71

(57) ABSTRACT

The present invention relates generally to methods for detecting complications, or abnormal conditions occurring during pregnancy, including placental abnormalities, eclampsia, or preelcampsia. The present invention comprises the steps of obtaining a sample from a patient, assaying the specimen to determine the level of bioactive lipids and comparing levels in the sample to control values or levels in normal samples, and correlating alterations to disease. The invention includes measuring panels of bioactive lipids to screen patients for disease and to monitor the progress of disease for diagnostic or therapeutic purposes.

### RELATED INFORMATION

This application is a continuation-in-part of co-pending application Ser. No. 09/585,138 filed on Jun. 1, 2000, U.S. Pat. No. 6,461,830. The priority of this prior application is expressly claimed, and the disclosure is hereby incorporated by reference in their entirety.

### FIELD OF INVENTION

The present invention relates to methods for the early detection of complications in pregnancy, including placental abnormalities and other conditions based on the detection of bioactive lipids. Specifically, the present invention relates to methods for early detection of pregnancy related disorders such as eclampsia and preeclampsia by detecting levels of bioactive lipids including phospholipids, sphingolipids, glycerophosphatidyl compounds, and lysophospholipids, as well as by-products and molecular species of these compounds in sample specimens obtained from pregnant women.

### BACKGROUND

Pregnancy is accompanied by a broad variety of physiological changes and complex metabolic processes that occur in the mother. These processes include lipid synthesis and several cellular mechanisms that involve lipid turnover. In some cases, pregnancy-related disorders affect lipid levels in the body and, when reflective of an underlying abnormality or disease, these lipid levels can be measured to detect the diseases. Many disorders that occur during pregnancy are also accompanied by abnormalities in the structure or function of the placenta including specifically eclampsia, preeclampsia, small gestational age, and others. The placenta is the key organ through which blood and nutrients flow from the mother to the infant and provides an essential lifeline for the developing fetus. When the placenta is abnormal, the result is a virtually unavoidable stress on the mother and infant.

Preeclampsia is an example of a disease accompanied by a placental abnormality and is responsible for up to 50-70% of hypertensive complications in pregnancies. Although eclampsia and preeclampsia are a leading cause of morbidity and mortality in pregnancies, the cause and etiology remain largely unknown. In severe cases, preeclampsia may develop into eclampsia, which often leads to death. Despite the dangers associated with preeclampsia, no cure exists, even although early detection and diagnosis enables therapy and treatment protocols that increase the chance to save the lives of the baby and the mother. Variations in severity are accompanied by a broad range of clinical treatment options, ranging from simple monitoring of the condition to aggressive therapy with specific pharmaceutical products. However, because of the difficulty in diagnosing the specific symptoms and severity of the disease, and the way the disease and symptoms may change over time making a prognosis or choosing a treatment option for preeclampsia and eclampsia is notoriously difficult.

Preeclampsia generally occurs after the 20^{th} week of pregnancy and appears without much warning. The most common symptoms are high blood pressure, swelling or edema of hands and feet, and increased protein in the urine. In the United States, preeclampsia is responsible for up to 10% of pregnancy-related mortality and morbidity each year. Preeclampsia is more prevalent in women under 20 or over 40 years of age. Those with pre-existing conditions of diabetes mellitus, renal diseases, high blood pressure, family history of preeclampsia, or previous complications with preeclampsia, are often at increased risk.

Placental abnormalities often accompany eclampsia and preeclampsia and also exist in several other complications of pregnancy, although in varying degrees of severity and at varying points during the term of the pregnancy. For example, in normal pregnancies, a certain type of cells, known as cytotrophoblast stem cells, invade the uterus to help exchange nutrients and oxygen between the mother and the fetus. In preeclamptic patients, cytotrophoblast stem cells develop abnormally and invade into the placenta only shallowly. This shallow invasion prevents adequate blood flow to the placenta and deprives normal oxygen and food flow to the fetus. Babies born to preeclamptic women are often underweight due to inadequate nutrition and availability of oxygen.

Depending on the severity of the placental abnormality or the preeclamptic condition, the overt symptoms vary from mild to severe. Mild preeclampsia is characterized by blood pressure readings of about 140/90 mm Hg, less than 5 g of protein in the urine a day, and swelling of face and hands. More severe forms of preeclampsia are characterized by blood pressure readings of about 160/110 mm Hg, over 5 g of protein in the urine a day, and beginning signs of end organ damage. Headaches, upper abdominal pains, impaired vision, fever, and vomiting are additional symptoms of preeclampsia. In extreme cases, preeclampsia can develop into eclampsia, which may lead to death and is characterized by seizure and coma. As noted above, the development of pregnancy-related disorders does not follow a recognizable course and the treatment and diagnosis are difficult because of variations in the symptoms and the severity of the disease. Additionally, the severity and type of placental abnormality can significantly impact the treatment and diagnosis of such a disorder and the progress of the disease can be erratic. Currently, there are no ideal methods to diagnose or monitor these disorders throughout pregnancy.

Even when early treatment and diagnosis is possible, pregnancy-related disorders are uniquely dangerous because of the altered physical state of the mother and the fragility of the fetus. To minimize risks to both mother and fetus, early detection and monitoring of these conditions throughout pregnancy is one of the most important factors in providing timely medical supervision and adequate expert care. Moreover, if the severity or likely course of the disease is misdiagnosed, unnecessary treatment also threatens the health of the mother and infant. Because the diagnosis and clinical assessment of patients with placental disorders and other pregnancy-related conditions are not precise, and varies from woman to woman, the need for continuous, accurate, and diagnosis throughout pregnancy is particularly great.

Moreover, the objective is to detect as many cases as possible at the earliest stage possible, it would is also be desirable to screen all mothers who are at risk for, or have a history of, pregnancy-related disorders. Thus, a pressing need exists for an accurate, easy and cost effective method of detecting pregnancy-related disorders, including placental abnormalities, preeclampsia, eclampsia, small for gestational age, pre-term labor, pregnancy-associated hypertension, pregnancy-associated diabetes and other diseases and conditions. Because the development of these diseases varies from patient to patient, and because the severity or prognosis of the disease may also vary during the course of the pregnancy, a reliable method for monitoring the development of a disease is also important.

### SUMMARY OF INVENTION

The present invention relates generally to methods for detecting complications in pregnancy, including diseases and disorders that are caused by or that accompany placental abnormalities, especially eclampsia and preeclampsia. The invention relies on the measurement of selected bioactive lipids, and preferably, a comparison of these lipids to values representing a normal or non-disease condition. Specifically, the present invention comprises the steps of obtaining a sample specimen from a patient, assaying the specimen to determine the level of one or more bioactive lipids, including phospholipids, glycerophosphatidyl compounds, glycerophosphatidylcholine, lysophospholipids, sphingolipids, and related compounds including specifically LPA, LPI, LPG, LPS, LPE, LPC, S-1-P, SPC, and molecular species of each in the sample, comparing levels in the sample to levels in control samples, and correlating alterations from the control value to the disease, prognosis, etc.

In a preferred embodiment of the diagnostic method of the invention, relative levels of molecular sub-species of one or more of the following compounds are measured: LPI, LPA, LPG, LPS, LPE, LPC, S-1-P, and SPC. Molecular sub-species contain varying lengths of hydrocarbon chains, and double bonds, e.g. 14:0, 16:0, 18:0, 18:1, 18:2, 20:4, and 22:6 sub-species. The selected analyte species may be compared to a normal value or as a portion of the total species, e.g. 18:0 LPA as a function of total LPA, and may be used independently, as an indicator of the presence or absence of a disease state, and may be compared with an earlier or later sample from the same patient. Changes in the relative levels of the analyte over time, or a compared to the normal value, or both, provide a prognosis and a correlation to a pregnancy-related disorder. In a preferred embodiment, discrete measurements are taken throughout a pregnancy, particularly in the first, second, or third trimesters—measurements may also be taken in specific time frames that are correlated to selected points in placental development, fetal development, or recognized changes in maternal lipid metabolism.

The invention includes several analytical methodologies, that are useful to perform the measurements needed to carry out the invention. Lipid measurements by several techniques are known, however, specific methods for measuring lipid species and sub-species using mass spectrometry (MS), liquid chromotography/mass spectrometry (LC/MS), or enzymatic reactions are particularly useful for these specific analytes. As described below, the individual measurements of lipid sub-species may be broken down into different components and analyzed numerically as rations, products, factors, and other measurements without departing from the spirit of the invention. Individual lipid species, including sub-species having varying hydrocarbon chains and degrees of saturation are preferably measured individually and collectively within a broader class of compounds. For example, total lysophospholipids (LPX) may be measured and used in a ratio that includes an individual lysophospholipid species such as LPA and/or as the individual component sub-species 14:0, 16:0, etc. Thus, the total LPA or LPI, for example, or any of the component molecular sub-species may be used in a ratio of the lipid species to the total lysophospholipids in a sample and this ratio and the component values may be tracked over time and compared to samples from the same patient or a subpopulation of patients exhibiting a certain disorder. With this methodology, the diagnosis of a pregnancy-related disorder is integrated into a methodology that monitors the progress of disease by measuring changes in selected bioactive lipid biomarkers that are indicative of disease.

In some cases, the relative amounts of the individual species may increase or decrease, either in relative or in absolute terms, both as a quantitative individual measurement or as a selected ratio. In one particular aspect of the invention, specific bioactive analytes maybe observed to be either elevated or lowered compared to normal at an early stage of disease, for example, prior to placental development, whereas after the onset of an abnormal placental development, the analyte is either elevated or lowered through the remainder of the pregnancy. In one particular embodiment, early stage disorders' exhibit lower levels of bioactive lipids while late stage disorders exhibit increased levels. In this embodiment, both the absolute and the relative measure of the concentration of an analyte is significant in the context of an overall profile for which varying levels of analyte or analyte ratios are known to be predictive of a certain disorder.

Depending on the analytical method, the specific analytes may be reacted in biochemical reactions that are designed to facilitate the measurement of specific analytes or specific analyte by-products. For example, the total amount of lysophospholipids (LPX) in a sample may be measured by conversion into glycerol-3-phosphate (G3P). In an especially preferred embodiment, the sample specimen is incubated with lysophospholipase and a non-specific glycerophsphoryl compound phosphodiesterase to produce G3P from LPX. Then, the concentration of G3P thus produced is thereafter determined using an enzymatic cycling reaction.

In another embodiment of the diagnostic method of the invention, the total amount of glycerophosphatidyl compounds (GPX) in a sample is measured by conversion into glycerol-3-phosphate (G3P). In an especially preferred embodiment, the sample specimen is incubated with a non-specific glycerophsphoryl compound phosphodiesterase to produce G3P from LPX. Then, the concentration of G3P thus produced is thereafter determined using an enzymatic cycling reaction.

In another preferred embodiment of the invention, the amount of lysophosphatidylcholine (LPC) in the sample is measured by enzymatically liberating choline from LPC. In this embodiment, samples are incubated with lysophospholipase and glycerophosphorylcholine phosphodiesterase to liberate choline from LPC. Choline is then preferably quantified using choline oxidase in a calorimetric reaction.

In another preferred embodiment of the invention, the amount of glycerophosphatidylcholine (GPC) in the sample is measured by enzymatically liberating choline from GPC. In this embodiment, samples are incubated with glycerophosphorylcholine phosphodiesterase to liberate choline from GPC. Choline is then preferably quantified using choline oxidase in a calorimetric reaction.

In yet another preferred embodiment of the invention, the amounts of at least two markers in the sample are measured, wherein the markers are chosen from the group consisting of glycerophosphatidyl compounds, glycerophosphatidylcholine, sphingolipids, and lysophospholipids, including, specifically, LPA, LPI, LPG, LPS, LPE, LPC, S-1-P, or SPC and individual, where present, molecular sub-species including 6:0, 14:0, 16:0, 18:0, 18:1, 18:2, 20:4, and 22:6 molecular sub-species.

Another aspect of the present invention concerns diagnostic kits for the determination of disease in pregnant patients according to the methods described herein. Preferred embodiments of the diagnostic kits include enzymes or other reagents necessary for the determination of the level of total glycerophosphatidyl compounds, sphingolipids, glycerophosphatidylcholine, or lysophospholipids in a specimen obtained from a pregnant patient, and instructions for making a diagnosis utilizing the kit. It is also preferred that the kit contain normal lipid level standards for comparison to the specimen obtained from the patient or certain values indicative of a normal or non-disease state.

### DESCRIPTION OF THE FIGURES

FIG. 1: A histogram of the GPX data shown in Table 1. The ● data points are control patients; the □ data points are preeclamptic patients.

FIG. 2: A histogram of the LPX data shown in Table 1. The ● data points are control patients; the □ data points are preeclamptic patients.

FIG. 3: A histogram of the GPC data shown in Table 2. The ● data points are control patients; the □ data points are preeclamptic patients.

FIG. 4: A histogram of the LPC data shown in Table 2. The ● data points are control patients; the □ data points are preeclamptic patients.

FIG. 5: FIG. 5 is the relative concentrations of molecular species of LPA in the three trimesters of pregnancy showing three individual determinations for both control values and for patient samples exhibiting preeclampsia.

FIG. 6: FIG. 6 is the relative concentrations of molecular species of LPI in the three trimesters of pregnancy showing three individual determinations for both control values and for patient samples exhibiting preeclampsia.

FIG. 7: FIG. 7 is the relative concentrations of molecular species of LPG in the three trimesters of pregnancy showing three individual determinations for both control values and for patient samples exhibiting preeclampsia.

FIG. 8: FIG. 8 is the relative concentrations of molecular species of LPS in the three trimesters of pregnancy showing three individual determinations for both control values and for patient samples exhibiting preeclampsia.

FIG. 9: FIG. 9 is the relative concentrations of molecular species of LPE in the three trimesters of pregnancy showing three individual determinations for both control values and for patient samples exhibiting preeclampsia.

FIG. 10: FIG. 10 is the relative concentrations of molecular species of LPC in the three trimesters of pregnancy showing three individual determinations for both control values and for patient samples exhibiting preeclampsia.

FIG. 11: FIG. 11 is the relative concentrations of sphingolipids S-1-P and SPC measured in each of the three trimesters of pregnancy for both control values and for patient samples exhibiting preeclampsia.

FIG. 12: FIG. 12 is the relative concentrations of the total lipid species of LPA, LPI, LPG, LPS, LPE and LPC for each of the three trimesters with individual determinations for both control values and for patient samples exhibiting preeclampsia.

### DETAILED DESCRIPTION

The present invention provides a simple, accurate, and minimally invasive method for detecting abnormal conditions in pregnancies. In the present invention, bioactive lipids, their precursors, molecular species, sub-species and metabolites in a patient sample are analyzed to determine the presence, absence, stage, prognosis, severity, or remission of a pregnancy-related disorder, preferably based on comparison with predetermined values indicating the absence of a disease, the presence of a specified stage of disease, a comparison with a previous diagnosis or an overall profile or panel measurement based on a series of measurements of bioactive lipids. Small amounts of bioactive lipids may be assayed accurately and reliably by traditional wet chemistry, by biochemical reactions, by mass spectrometry, or mass spectrometry in combination with other analytical techniques, such as LC/MS. Bioactive lipids analyzed in the present invention comprise one or more lipids selected from the lysophospholipid (LPX) and sphingolipid (SPX) families, including sphingosine-1-phosphate (S-1-P), sphingosylphosphorylcholine (SPC), and sphingomyelin. As used herein, the term bioactive lipids also encompasses precursors or metabolites of those lipids, such as glycerophosphitidyl (GPX) compounds, although they are not technically lipids. Glycerophosphatidyl compounds, as the term is used in the present invention, include glycerol-3-phoaphate (G3P), glycerophosphatidylinositol (GPI), glycerophosphatidylcholine (GPC), glycerophosphatidylserine (GPS), glycerophosphatidylglycerol (GPG), and glycerophosphatidylethanolamine (GPE). Bioactive lipid precursors may also be measured in the methods of the invention.

LPX species for analysis pursuant to the invention include, among others, lysophosphatidic acid (LPA), lysophosphatidyl choline (LPC), lysophosphatidyl serine (LPS), lysophosphatidyl ethanolamine (LPE), lysophosphatidyl inositol (LPI), and lysophosphatidyl glycerol (LPG). Lysophospholipids have the general structure of a glycerol backbone with a phosphate or a derivatized phosphate such as choline, inositol, ethanolamine, glycerol, or serine at the sn-3 position; a single hydrocarbon chain located at the sn-1 or sn-2 position, linked to the glycerol backbone by an acyl linkage; and a hydroxyl at the unoccupied sn-1 or sn-2 position. Alternatively, a hydrocarbon chain is linked to the glycerol backbone at the sn-1 or the sn-2 position by an alkyl or alkenyl linkage with a hydroxyl at the unoccupied sn-1 or sn-2 position. Alternatively, an acetyl chain or other short hydrocarbon chain is linked to the glycerol backbone at the sn-1 or sn-2 position. These compounds have the following general structures:

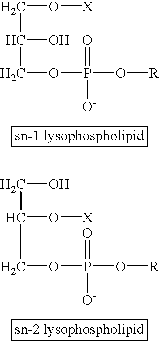

where X is any fatty acid, alkyl, or alkenyl chain. Such carbon chains include, but are not limited to 6:0, 14:0, 18:0, 16:0, 18:1, 18:2, 20:4n-6 and 22:6n-3, attached through an acyl (G—O—COOR1), alkyl (G—O—CH₂—R1) or alkenyl bond (G—O—CH═R1),

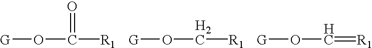

where “G” is the glycerol backbone. X may include such fatty acids as palmitic, palmitoleic, stearic, oleic, linoleic, arachidonic, and docasahexahoic fatty acid linked. R can be any phosphate derivative moiety, including, but not limited to, hydrogen, choline, inositol, ethanolamine, glycerol, and serine. More generally, the lysophospholipids may be be represented by the structural formula:

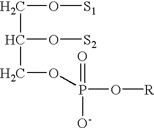

wherein S₁ is either X (as defined above) or hydrogen; S₂ is hydrogen if S₁ is X, or X if S₁ is hydrogen; and R is as defined above. Common lysophospholipids for detection using the invention method include, but are not limited to, LPA, LPC, LPS, LPE, LPI, and LPG, and their specific fatty acid/alcohol side chain forms.

Sphingolipids contemplated for analysis in the present invention comprise, among others, sphinganine-1-phosphate, sphingosine-1-phosphate (S1P), sphingosylphosphorylcholine (SPC), and sphingomyelin. The structural formulae for these sphingosyl compounds are shown below: Sphingosine-1-Phosphate:

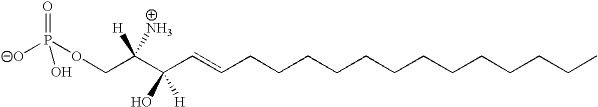

Sphinganine-1-Phosphate:

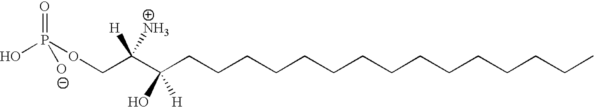

Sphingomyelin:

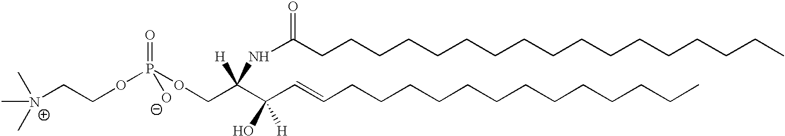

Sphingosine-phosphorylcholine (SPC):

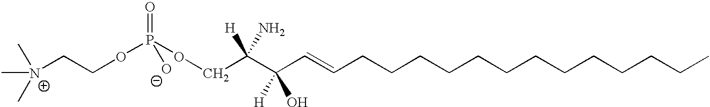

Thus, sphingosyl compounds, which are the sphingomyelin analogs of lysophospholipids, have the basic structural formula:

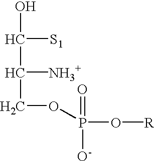

wherein S₁ is any alkyl or alkenyl of 15 carbons, and wherein R is any phosphate derivative moiety, including, but not limited to, hydrogen, choline, inositol, ethanolamine, glycerol, and serine. Thus, in addition to the derivative SPC, sphingosylphosphorylinositol (SPI), sphingosylphosphorylethanolamine (SPE), sphingosylphosphorylglycerol (SPG), and sphingosylphosphorylserine (SPS) are also suitable bioactive lipid analytes for measurement in the methods of the present invention. By convention, 1-unsaturated S₁ sphingosyl compounds are called “sphingosine” compounds, and saturated S₁ sphingosyl compounds are called “sphinganine” compounds.

In addition to the bioactive lipids described above, precursors and metabolites of bioactive lipids may also be assayed. Precursors include, among others, one or more of phospholipids (2 fatty acid chains), glycerophosphatidyl compounds (GPX), monoglyceride (MG), and digycleride (DG). Metabolites comprise, among others, one or more of glycerophospholipids (GPX), and monoglyceride (MG). Glycerophosphatidyl compounds (GPX) suitable for assaying in the methods of the present invention comprise, among others, glycerol-3-phosphate (G3P), glycerophosphatidyl inositol (GPI), glycerophosphatidyl choline (GPC), glycerophosphatidyl serine (GPS), glycerophosphatidyl glycerol (GPG), and glycerophosphatidyl ethanolamine (GPE). These compounds are metabolically related to lysophospholipids, and have the basic structure above, wherein both S₁ and S₂ are hydrogen. In addition, bioactive lipid “R” groups may be measured, such as choline, serine, ethanolamine, inositol, glycerol, and phosphate derivatives of these groups. GPX, MG, DG, and the derivative groups may constitute either a precursor or a metabolite. Additional precursors and metabolites not specifically mentioned herein are well known to one of ordinary skill in the art.

To ascertain absolute or relative levels of bioactive lipids which are indicative of pregnancy-related disorders, samples obtained from symptomatic or asymptomatic patients may be analyzed for comparison with a standard solution or normal level numerical values indicative of the absence of disease, or preset values that indicate a specific diagnosis of a disease. The disease diagnosis may be also correlated to the duration of the pregnancy, specifically the first, second or third trimester, or the state of placental development that ordinarily accompanies a pregnancy of a specified duration. By comparing the absolute or relative levels of bioactive lipids, an individual value, ratio of values or multi-value or multi-ratio profile of bioactive lipids can be developed to correlate the date with the presence or absence of disease. Significant deviation from a standard or normal level (meaning a deviation greater that seen in pregnancies which complete term without the disorder) may be indicative of a decreased probability of the infant developing to term. In preferred embodiments, this standard level may be determined empirically from a statistically significant population of successful pregnancies and can be expressed as a number with a standard deviation. The standard level may be the median level of the bioactive lipid for normal pregnancies or may be derived through some other statistically valid means. This empirical approach will be useful where the detection mechanism is sufficiently accurate to yield reproducible quantitative results. In another embodiment, the standard level for comparison may be a sample that is spiked with bioactive lipids to the standard level. By comparing the types of bioactive lipids and their concentrations to the standard levels, a determination may be made as to the likelihood of whether the pregnancy continuing successfully to term. As noted above, embodiments of the present invention, the level of a single bioactive lipid may be detected, or, two or more bioactive lipids, species, or sub-species may be detected simultaneously. The levels may also be expressed as a ratio of individual species or as a single or multiple species ratio to lipid class, e.g. LPA/LPX, LPC/LPX, and LPI/LPX, etc.

In alternative embodiments, multiple specimens may be collected over a time interval, and the change in bioactive lipid levels over the course of the time interval determined. This value may be compared to a standard change in the level of the bioactive lipids assayed, obtained in a manner similar to that described above for the standard level, wherein multiple samples are collected from the IVF embryo cultures over a similar time interval. Similar to the analysis above, a significant deviation in the change observed in the sample indicates a change in the lipid metabolism occurring during the pregnancy. Thus, the invention analyzes one or more bioactive lipids, correlates that measurement to a standard that has been predetermined or is determined according to the practice of the invention, and then determines a status of the pregnancy based on a determination of the selected levels of bioactive lipids and a known correlation to a disorder or abnormality in placental development.

The determination of the desirable biological characteristic may be expressed in terms of a “bright line test” or a probability or other gradient measure. For example, in a bright line test, if a specific bioactive lipid is present at a zero or undetectable level in the sample, or below a definite threshold, the pregnancy-related disorder embryo is deemed not to exist, whereas if the bioactive lipid is present at detectable levels or above a certain threshold, the disorder is present or subject to verification by other diagnostic or clinical testing. As will be appreciated by those of skill in the art, the analysis or measurement of the bioactive lipids can be conducted by determining an increase or decrease from a pre-existing standard, an increase or decrease from a previous measurement, or an absolute value relative to zero or a standard threshold or variable concentration established through testing or experience.

Several analytical techniques may be used for detecting and measuring bioactive lipids, including mass spectrometry procedures, nuclear magnetic resonance (NMR) procedures, and enzymatic methods to detect or quantitatively measure bioactive lipids in a sample specimen. As noted above, a preferred embodiment of the invention, liquid chromatography—mass spectrometry (LC-MS) is used to analyze a patient sample specimen for bioactive lipids and sub-species of lipids. Samples from pregnant women are diluted in a suitable solvent (e.g., 1:5 dilution in methanol) and centrifuged to remove any precipitate. In some embodiments, protein-lipid interactions may be disrupted by adding acid, or other substances, to the sample. The samples are separated on an LC or high pressure (HP) LC column, and the individual bioactive lipids detected and quantified by mass spectroscopy. In a preferred embodiment, electrospray (ESI) MS or atmospheric pressure chemical ionization (APCI) MS is used. The mass spectroscopy technique is very flexible, and may be used to measure lysophospholipids, sphingolipids, and glycerophosphatidyl compounds, as well as to distinguish between various fatty acid or long-chain alcohol side chains and phosphatidyl substituents within the general species.

In one embodiment, the bioactive lipids are assayed enzymatically. The enzymatic assay measures the total concentration of lysophospholipids or glycerophosphatidyl compounds present in a sample, and may also be used to measure the concentrations of specific bioactive lipids. The enzymatic assay preferably measures total GPX and/or LPX in the sample. The specification of U.S. Pat. No. 6,248,553, ENZYME METHOD FOR DETECTING LYSOPHOSPHOLIPIDS AND PHOSPHOLIPIDS AND FOR DETECTING AND CORRELATING CONDITIONS ASSOCIATED WITH ALTERED LEVELS OF LYSOPHOSPHOLIPIDS, the specification of U.S. Pat. No. 6,255,063, ENZYME METHOD FOR DETECTING LYSOPHOSPHOLIPIDS AND PHOSPHOLIPIDS AND FOR DETECTING AND CORRELATING CONDITIONS ASSOCIATED WITH ALTERED LEVELS OF LYSOPHOSPHOLIPIDS, filed May 15, 1999, and PCT Publication No. WO 00/23612, which is instructive for teaching methods of measuring LPX, LPA and LPC levels, are fully incorporated herein by reference. In addition, the specification of U.S. application serial No. 09/558,880, METHOD OF DETECTING CARCINOMAS, filed Apr. 26, 2000, which is instructive for teaching methods of measuring GPX, G3P, and GPC levels, is also fully incorporated herein by reference.

A preferred method for enzymatically measuring bioactive lipids comprises measuring GPX and LPX in the specimen. The analytic method generally comprises converting GPX or LPX into G3P and assaying for the concentration of G3P produced in the sample. To convert LPX into G3P, lysophospholipase is used in the enzymatic reaction to cleave the fatty acid group from the G3P and other glycerophosphatidyl compound (GPX) backbones. GPX is preferably digested using glycerophosphatidyl compound phosphodiesterase (GPX-PDE) to cleave the substituent from the phosphate of the G3P backbone. Thus, the amount of G3P produced by both of these enzymatic cleavage reactions is directly proportional to the total amount of LPX in the media specimen. Similarly, to determine the amount of GPX in a sample, only the glycerophosphatidyl compound phosphodiesterase is used to cleave the phosphatidyl substituents from the GPX species.

The amount of G3P in the cleaved and uncleaved portions of the sample specimen is then quantified using conventional or enzymatic techniques. If the size of the media specimen is 2 ml or less, a quantification technique capable of detecting picomole amounts of the glycero compound is used. Suitable conventional techniques for detecting picomole amounts include mass spectrometry.

Another preferred technique for determining the amount of G3P in the samples is an enzymatic cycling reaction. Specifically, an enzyme cycling reaction using glycerol-3-phosphate dehydrogenase (GDH), glycerol-3-phosphate oxidase (GPO) and NADH is used to accumulate H₂O₂ and NAD. In the reaction, G3P is converted into dihydroxyacetone phosphate (DAP) and H₂O₂ using GPO in the presence of oxygen and water. In the presence of DAP, G3P dehydrogenase converts dihydroxyacetone phosphate back to G3P and oxidizes NADH to NAD. Alternatively, depending on the sensitivity of the NADH or H₂O₂ detection, a non-cycling reaction may be used.

The disappearance of NADH is monitored spectrophotometrically preferably at OD₃₄₀. In alternative embodiments, H₂O₂ production may be measured by colorimetry, fluorometry, or by chemiluminescence. For the colorimetric assay, any of a number of chromogenic substrates, such as 4-aminoantipyrine (AAP), pyrogallol, 2-(2¹-Azinobis (3-ethylbenzthiazoline-sulfonic acid)(ABTS) and 3,3¹,5,5¹-tetramethylbenzidine) (TMB), may be used with a peroxidase to generate detectable signal (e.g., OD₅₀₅ for AAP). Numerical values are obtained from a standard curve consisting of known concentrations of G3P, and assays are preferably performed in duplicate with both positive and negative controls. The difference between the detectable signal (e.g., OD₃₄₀ or OD₅₀₅) before and after the enzyme cycling reaction is directly proportional to the amount of G3P present.

Additional bioactive lipids, such as glycerol-3-phosphate (G3P) and lysophosphatidic acid (LPA), may also be determined as above by first separating LPA and G3P from the total lysophospholipid in the sample. In a preferred embodiment for determining the concentration of LPA and G3P, G3P is measured after being liberated from LPA in the absence of GPX-PDE. In this embodiment, G3P is detected using the enzymatic cycling reaction described above, but the remaining GPX compounds other than G3P are not detected. LPA is first cleaved into glycerol-3-phosphate and fatty acid using phospholipase B or lysophospholipase. The level of G3P is then measured using G3P dehydrogenase and oxidase in the cycling reaction as described above.

Additional bioactive lipids, such as glycerophosphatidylcholine and lysophosphatidylcholine, may also be determined as above by first separating LPC and GPC from the total lysophospholipid in the sample. In a preferred embodiment for determining the concentration of LPC and GPC, choline is measured after being liberated from GPC and LPC. LPC is first cleaved into glycerophosphatidyl choline and fatty acid using phospholipase B or lysophospholipase. The level of LPC is then determined by liberating choline and glycero-3-phosphate (G3P) from glycerophosphorylcholine using glycerophosphorylcholine phosphodiesterase (GPC-PDE), followed by a colorimetric enzymatic determination of choline using choline oxidase, 4-aminoantipyrine (AAP), 3,5 Dichloro-2-hydroxybenzenesulfonic acid sodium salt (HDCBS) and peroxidase. Choline is preferably detected by oxidizing to H₂O₂ and betaine and using peroxidase to form quinoneimine dye. Alternatively, G3P is measured using G3P dehydrogenase and oxidase in the cycling reaction as described above.

In other embodiments of the invention, immunoassay techniques may be utilized to measure the levels of bioactive lipids in the pregnant patient sample. Antibodies to various biological lipids suitable for analysis have been described in the literature. For example, Chen, et al., “Production and Application of LPA Polyclonal Antibody,” Bioorg. & Med. Chem. Letters 10:1691-1693 (2000), describes the production and use of an antibody to LPA, utilizing colloidal gold as an antigen carrier. Similar antibodies may be raised to other lysophospholipid species and utilized in immunoassay formats (e.g., sandwich or competitive immunoassays.) In addition, antibodies to bioactive lipid precursors or metabolites have been reported. For example, Echelon Labs (Salt Lake City, Utah) offers an anti-phosphoinositol antibodies, and immunoassay kits for the detection of phosphoinositol species. One of ordinary skill in the art would be able to readily adapt these antibodies for analysis of bioactive lipids in the pregnant patient sample.

To optimize detection of lysophospholipids, inhibitors may be used to prevent degradation of the glycerophosphatidyl compounds and lysophospholipids in the sample. Such inhibitors include phosphodiesterase inhibitors such as IBMX (3-Isobutyl-1-methylxanthine, CalBiochem, La Jolla, Calif.); Ro-20-1724 (CalBiochem); Zaprinast (CalBiochem) and Pentoxifylline (CalBiochem); general protease inhibitors such as E-64 (trans-Epoxysuccinyl-L-leucylamido-(4-guanidino)butane, Sigma); leupeptin (Sigma); pepstatin A (Sigma); TPCK (N-tosyl-L-phenylalanine chloromethyl ketone, Sigma); PMSF (Phenylmethanesulfonyl fluoride, Sigma); benzamidine (Sigma) and 1,10-phenanthroline (Sigma); organic solvents including chloroform and methanol; detergents such as SDS or Trident X100; proteases that would degrade phospholipases such as trypsin (Sigma) and thermostable protease (Boehringer Mannheim Biochemicals, Indianapolis, Ind.); and metal chelators such as EDTA (Ethylenediaminetetracetic acid, Sigma) and EGTA (Ethylene glycol-bis-(beta-aminoethyl ether), Sigma). In some embodiments, MgCl₂ and/or EDTA are included in the assay buffers to optimally determine levels of each analyte.

In a preferred embodiment, microtiter plates may be used for small volumes of samples and reagents. An ELISA reader may also be used to monitor and help automate the assay, and the reduced processing times may in turn reduce variability between results. Also, micro-scale automated assay equipment, such as the Immuno I system available from Bayer, the Access system available from Beckman Coulter, or the Dimension RxL HM system available from Dade Behring may be used.

The present invention also contemplates convenient pre-packaged diagnostic kits for enzymatically detecting levels of bioactive lipids. Preferably, these kits contain enzymes and reagents necessary for determining the concentrations of lipids and/or molecular species, as well as instructions to correlate the bioactive lipid levels assayed using the kits to complications of pregnancy, specifically placental abnormalities, and more specifically eclampsia or preeclampsia. For example, diagnostic kits preferably include enzymes and buffers for the cleavage of GPX, GPC, LPX and LPC. Exemplary enzymes for inclusion in such kits are phospholipase B, lysophospholipase, glycerophosphitidyl compound phosphodiesterase, and glycerophosphatidylcholine phosphodiesterase. In addition, the kits of the present invention preferably include reagents for determining concentrations of G3P, including enzymatic reaction reagents such as glycerol-3-phosphase dehydrogenase, glycerol-3-phosphate oxidase, NADH and other ancillary agents such as buffering agents, colorimetric reagents for the detection of peroxide generation, and EDTA for inhibiting degradation of G3P.

Optionally, the kits may include reagents necessary to separate GPC or LPC from the other lysophospholipids in the sample. Also optionally, the kits of the present invention include reagents for measuring choline liberated from GPC or LPC in the specimen. Such reagents may include, for example, choline oxidase, peroxidase, 4-aminoantipyrine (AAP), 3,5 Dichloro-2-hydroxybenzenesulfonic acid sodium salt (HDCBS), and other ancillary agents such as buffering agents.

The kits of the invention also comprise containers and measuring apparatus for carrying out the appropriate measurements. The kits of the invention may also include standards for comparison. In order to assure that the clinician has properly performed the analysis described herein when using the kit control standards may also be provided. Variations of specific container and combination embodiments of the kits of the invention may readily be devised by those of ordinary skill in the art utilizing the guidance herein provided.

As noted above, the present invention may be used throughout the various stages of pregnancy. Preferably, the measurement of bioacive lipids begins during the first trimester of pregnancy in order to start therapeutic interventions as early as possible. However, the methods of the invention is also used in the second or third trimester of pregnancy, especially where symptoms appear with placental development at approximately 8-12 weeks. The present invention is also minimally invasive and cost efficient. Prepackaged diagnostic kits for measuring levels of glycerophosphatidyl compounds, glycerophosphatidylcholine, lysophospholipids and lysophosphatidylcholine in specimens from pregnant patients, as described below, can easily be used by clinicians for widespread screening of pregnant patients for preeclampsia. Thus, the present invention provides an easily administered test for a major health threat to pregnant women.

Different types of specimens, including plasma, serum, and urine, may be used in the claimed methods for detecting preeclampsia. Anti-coagulants such as heparin and chelating agents are usually added to whole blood specimen to minimize the activation of platelets and to reduce endogenous enzymatic activity. In obtaining a serum specimen, whole blood is preferably centrifuged by standard procedures at 500×g for 3 minutes or up to 3000×g for 15 minutes. A plasma sample is typically obtained by centrifugation. The blood sample is centrifuged at preferred speeds of between 400 to 1400×g to pellet out the blood cells, and the resulting supernatant is collected. Higher speeds of 2000 to 3000×g may also be used to more thoroughly pellet out the platelets. Further, urine specimens may be collected under conventional conditions.

Depending on the type of specimen and preparation procedure employed, the particular level of the bioactive lipid indicative of a pregnancy related disorder may vary. Although the data presented below illustrate appropriate levels to differentiate between normal and preeclamptic levels of several bioactive lipids in each of the different specimen types, separate parameters may be developed by the practitioner using routine experimentation as guided by the specification and the examples below. Initially, levels of both normal and disease state concentrations of bioactive lipids are determined using normal, non-disease state samples for the type of specimen, as has been done for plasma and serum samples in the Examples below. By analyzing this data, one of ordinary skill in the art may determine the particular concentration of bioactive lipid in a sample type which signals, for example, a significant decrease in levels of GPX, or an increase in LPX in the sample as compared to normal levels of the same sample type. Then, this data analysis may be used for comparison with a specimen obtained from a patient to determine whether the level in the specimen is significantly higher than normal, lower than normal, or altered from a previous assay of the same patient, thereby indicating preeclampsia, a placental abnormality, or other pregnancy-related disorder.

In one preferred embodiment, combinations of bioactive lipids are simultaneously assayed in a sample from a patient, and the levels of these molecules are used in conjunction to arrive at a diagnosis of a pregnancy-related disorder, particularly preeclampsia. As is evident in the data presented in Tables 1 and 2, normal patients present a wide range of GPX, GPC, LPX and LPC levels, although preeclamptic patients present levels which are more consistently low. By manipulating the data obtained from assessing GPX, GPC, LPX and LPC, a practitioner in the medical diagnostic arts can devise combination tests utilizing these data which will eliminate some of the normal variance in individual GPX, GPC, LPX and LPC levels and allow for a better separation of non-preeclamptic from preeclamptic patients. For instance, assaying combined GPX+LPX levels may allow one to distinguish non-preeclamptic patients from preeclamptic patients (by generating fewer “false positives” below the preeclamptic indication level) better than assaying patients for GPX or LPX alone. An added benefit to such combined level analyses is that a one well reaction which cleaves both LPX and GPX (or LPC and GPC) may be used, rather than the two wells which are necessary to measure LPX and GPX (or LPC and GPC) separately, as described below.

In an alternative embodiment of the present invention, an individual is tested repeatedly over time to monitor for any significant changes. A significant change such as a decrease in GPX, GPC, LPX or LPC concentrations as compared to previous levels in a specimen from the patient may signal the onset of preeclampsia, or further deterioration of the preeclamptic condition. In contrast, increases in levels of GPX, GPC, LPX or LPC closer to normal levels may signal an improvement in the condition.

In practice, the comparison of a value from a patient sample to a control value may be achieved by using a simple numerical reference control value that is based on a population of samples reflecting a normal physiology that has been provided with the assay of the present invention for comparative purposes such that a comparison to the assay value can be made and the differential used to assess the preeclamptic condition. Alternatively, the control value may be obtained by performing the assay in duplicate with a control that contains one or more predetermined concentration or concentrations (i.e. to determine linearity) of any of the above-described compounds. In certain assay systems, the control value may be a calibration standard obtained from a control solution. In the embodiment of the invention where the patient's condition is monitored over time, the control value may comprise, or be compared to, a reading from the same patient under a different condition, particularly at a different point in time. With this approach, the condition can be monitored over a period of time by obtaining a series of samples and measurements for comparison both with control value(s) and with previous measurements.

A preferred method for measuring GPX and LPX in the specimen generally comprises converting GPX or LPX into G3P and assaying for the concentration of G3P produced in the sample. A portion of the specimen, which has not been enzymatically converted, may also be assayed for the concentration of endogenous, or “background” G3P. Otherwise, the level of all glycerophosphatidyl compounds is measured after conversion of GPX to G3P. Thus, the amount of G3P produced by the enzymatic cleavage of LPX may be determined by subtracting the level of “background” G3P and G3P produced from GPX from the G3P detected in the phospholipase cleaved sample.

To convert LPX into G3P, lysophospholipase is used in the enzymatic reaction to cleave the fatty acid group from the G3P and other glycerophosphatidyl compound (GPX) backbones. GPX is preferably digested using glycerophosphatidyl compound phosphodiesterase (GPX-PDE) to cleave the substituent from the phosphate of the G3P backbone. Thus, the amount of G3P produced by both of these enzymatic cleavage reactions is directly proportional to the total amount of LPX in the specimen, and the amount of G3P produced from GPX-PDE cleavage alone is directly proportional to the amount of GPX in the sample.

The amount of G3P in the cleaved and uncleaved portions of the sample specimen is then quantified using conventional or enzymatic techniques. If the size of the blood specimen is 2 ml or less, a quantification technique capable of detecting picomole amounts of the glycero compound may be used. Suitable conventional techniques for detecting picomole amounts include mass spectrometry.

Another preferred technique for determining the amount of G3P in the samples is an enzymatic cycling reaction. Specifically, an enzyme cycling reaction using glycerol-3-phosphate dehydrogenase (GDH), glycerol-3-phosphate oxidase (GPO) and NADH is used to accumulate H₂O₂ and NAD (U.S. Pat. No. 5,122,454, Ueda et al.). In the reaction, G3P is converted into dihydroxyacetone phosphate (DAP) and H₂O₂ using GPO in the presence of oxygen and water. In the presence of DAP, G3P dehydrogenase converts dihydroxyacetone phosphate back to G3P and oxidizes NADH to NAD.

The disappearance of NADH is monitored spectrophotometrically preferably at OD₃₄₀. In alternative embodiments, H₂O₂ production may be measured by colorimetry, fluorometry, or chemiluminescence. For the colorimetric assay, any of a number of chromogenic substrates, such as 4-aminoantipyrine (AAP), pyrogallol, 2-(2¹-Azinobis (3-ethylbenzthiazoline-sulfonic acid)(ABTS) and 3,3¹,5,5¹-tetramethylbenzidine) (TMB), may be used. Numerical values are obtained from a standard curve consisting of known concentrations of G3P, and assays are preferably performed in duplicate with both positive and negative controls. The difference between OD₃₄₀ or OD₅₀₅ before and after the enzyme cycling reaction is directly proportional to the amount of G3P present. Background signals in the specimen without the cycling enzyme mix are subtracted from all samples, and G3P standard curve values are plotted and fitted to a linear or second-order polynomial curve fit. The levels of G3P in each sample are determined by comparing each signal measured to the standard curve. Finally, the level of G3P attributable to LPX is determined by subtracting out the GPX measurement. Optionally, one can determined the level of substituted glycerophosphatidyl compounds (GPC, GPI, GPE, GPG, GPS) by subtracting out the G3P level detected in a portion of the sample which has not been digested with a phospholipase or GPX-PDE.

Although the level of glycerophosphatidylcholine and lysophosphatidylcholine may be determined by as above by first separating LPC and GPC from the total lysophospholipid in the sample, in a preferred embodiment for determining the concentration of LPC and GPC, choline is measured after being liberated from GPC and LPC. Glycerophosphorylcholine and fatty acid are first liberated from LPC using phospholipase B or lysophospholipase. The level of LPC is then determined by liberating choline and glycero-3-phosphate (G3P) from glycerophosphorylcholine using glycerophosphorylcholine phosphodiesterase (GPC-PDE), followed by a calorimetric enzymatic determination of choline using choline oxidase, 4-aminoantipyrine (AAP), 3,5 Dichloro-2-hydroxybenzenesulfonic acid sodium salt (HDCBS) and peroxidase. The background level of glycerophosphorylcholine and choline is determined by measuring the glycerophosphorylcholine and choline in a portion of the sample which has not been enzymatically treated with phospholipase to cleave LPC. Although the background level of endogenous choline may be determined by performing the choline oxidase reaction in a portion of the sample which has not been digested with a phospholipase or GPC-PDE, the amount of endogenous choline in a sample was not significant in the applicant's experience.

Choline is preferably detected by oxidizing to H₂O₂ and betaine and using peroxidase to form quinoneimine dye. Alternatively, G3P is measured using G3P dehydrogenase and oxidase in the cycling reaction as described above. After comparison to a standard curve, the level of LPC in the sample is determined by subtracting the background level of glycerophosphorylcholine and choline from the level of choline determined in the phospholipase cleaved portion of the sample.

To optimize detection of lysophospholipids, inhibitors may be used to prevent degradation of the glycerophosphatidyl compounds and lysophospholipids in the sample. Such inhibitors include phosphodiesterase inhibitors such as IBMX (3-Isobutyl-1-methylxanthine, CalBiochem, La Jolla, Calif.); Ro-20-1724 (CalBiochem); Zaprinast (CalBiochem) and Pentoxifylline (CalBiochem); general protease inhibitors such as E-64 (trans-Epoxysuccinyl-L-leucylamido-(4-guanidino)butane, Sigma); leupeptin (Sigma); pepstatin A (Sigma); TPCK (N-tosyl-L-phenylalanine chloromethyl ketone, Sigma); PMSF (Phenylmethanesulfonyl fluoride, Sigma); benzamidine (Sigma) and 1,10-phenanthroline (Sigma); organic solvents including chloroform and methanol; detergents such as SDS or Trident X100; proteases that would degrade phospholipases such as trypsin (Sigma) and thermostable protease (Boehringer Mannheim Biochemicals, Indianapolis, Ind.); and metal chelators such as EDTA (Ethylenediaminetetracetic acid, Sigma) and EGTA (Ethylene glycol-bis-(beta-aminoethyl ether), Sigma). In some embodiments, MgCl₂ and/or EDTA were included in the assay buffers to optimally determine levels of each analyte.

In a preferred embodiment, microtiter plates may be used for small volumes of samples and reagents. An ELISA reader may also be used to monitor and help automate the assay, and the reduced processing times may in turn reduce variability between results. The methods of the present invention may further be easily adapted for use in micro-scale automated assay equipment, such as the Immuno I system available from Bayer, the Access system available from Beckman Coulter, or the Dimension RxL HM system available from Dade Behring.

The present invention also contemplates convenient pre-packaged diagnostic kits for detecting levels of GPX, GPC, LPX and/or LPC. Preferably, these kits contain enzymes and reagents necessary for determining the level of GPX, GPC, LPX and/or LPC as described above. For example, diagnostic kits preferably include enzymes and buffers for the cleavage of GPX, GPC, LPX and LPC. Exemplary enzymes for inclusion in such kits are phospholipase B, lysophospholipase, glycerophosphitidyl compound phosphodiesterase, and glycerophosphatidylcholine phosphodiesterase. In addition, the kits of the present invention preferably include reagents for determining concentrations of G3P, including enzymatic cycling reaction reagents such as glycerol-3-phosphase dehydrogenase, glycerol-3-phosphate oxidase, NADH and other ancillary agents such as buffering agents, colorimetric reagents for the detection of peroxide generation, and EDTA.

Optionally, the kits may include reagents necessary to separate GPC or LPC from the other lysophospholipids in the sample. Preferably, the kits of the present invention include reagents for measuring choline liberated from GPC or LPC in the specimen. Such reagents may include, for example, choline oxidase, peroxidase, 4-aminoantipyrine (AAP), 3,5 Dichloro-2-hydroxybenzenesulfonic acid sodium salt (HDCBS), and other ancillary agents such as buffering agents.

The kits of the invention also comprise containers and appropriate instructions for carrying out the inventive method. Preferred embodiments of the kits of the invention also include standards for comparison with the specimens obtained from the patients, in order to assure that the clinician has properly performed the claimed method while using the kit. Variations of specific container and combination embodiments of the kits of the invention may readily be devised by those of ordinary skill in the art utilizing the guidance herein provided.

The present invention is further described in the following examples. These examples do not, in any way, limit the present invention.

### EXAMPLES

### Example 1

### Enzyme Cycling Assay of Plasma Specimen Levels of Lysophospholipid (LPX) and Glycerophosphatidyl Compounds (GPX) as Measured by Levels of G3P for the Detection of Preeclampsia

Plasma samples were obtained from blood specimen provided by twenty female patients. A whole blood specimen was collected from each of the patients in a vacutainer tube containing EDTA. The whole blood specimen was then centrifuged under standard conditions to provide a pellet of the blood cells and platelets and a supernatant. The plasma supernatant was either processed immediately or stored at −70° C.

Reagents

Lysophospholipase (LYPL) was purchased from Asahi Chemical Industry, Tokyo, Japan. Glycerol-3-phosphate oxidase, glycerol-3-phosphate dehydrogenase, human plasma, human serum, 4-aminoantipyrine (AAP), and glycerophosphorylcholine phosphodiesterase (GPX-PDE) were purchase from Sigma Chemical Co., St. Louis, Mo. Peroxidase and NADH were purchased from Boehringer Mannheim, Indianapolis, Ind. All lipid or glycerophosphatidyl standards were purchased from Avanti Polar Lipids, Alabaster, Ala. or Sigma Chemical Co.

Enzyme Assay

Using a 96 well microtiter plate, 5 μl of the diluted sample were aliquotted into pairs of wells. To one well of each pair, the “LPX+GPX” well, 100 μl of LYPL (0.05 Units)/GPC-PDE (0.0125 Units) were added. 100 μl of GPC-PDE (0.0125 Units) were added to the other “background GPX” well. The wells were then incubated at 37° C. for 15 minutes. Glycerophosphatidyl compounds were produced as an intermediate by LYPL digestion of LPX. G3P and the phosphoryl substituents were then liberated from the glycerophosphatidyl compounds using GPX-PDE. G3P levels were then determined by enzymatic assay of the plasma samples. 100 μL of cycling reaction enzyme mix containing 10 units of G3P dehydrogenase, 4 units of G3P oxidase, and 0.34 mM NADH in 50 mM Tris (pH 8.0) were added to each well, and incubated at 37° C. for 30 minutes. The G3P oxidase converts G3P to dihydroxyacetone phosphate and H₂O₂, and G3P dehydrogenase converts the dihydroxyacetone phosphate back into G3P. This reaction oxidizes NADH to NAD, and as cycling continues, both H₂O₂ and NAD accumulate.

The total amount of G3P was determined by monitoring the oxidation of NADH (i.e. the reduction of absorbance at 340 nm after the cycling action compared to A₃₄₀ before cycling). In addition, the accumulation of H₂O₂ was determined colorimetrically by adding 50 μl of a solution containing 0.5 units peroxidase, 0.5% HDCBS and 0. 15% AAP in 50 mM Tris 8.0 to each well and recording the absorbance at 505 nm.

Numerical values of G3P concentrations were obtained from a standard curve constructed from known G3P amounts. An internal standard of plasma was included within each assay (i.e. each plate) that was measured at different dilutions. In some cases, this internal standard was used to correct for variations between different experiments. When the colorimetric method was used, the plate was blanked at 505 nm prior to color development.

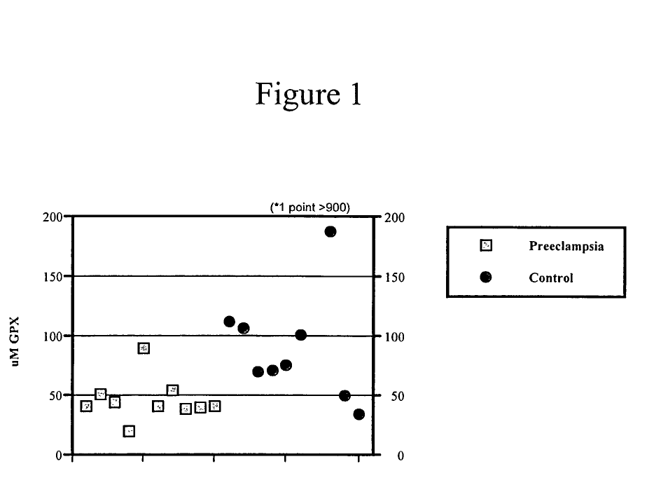

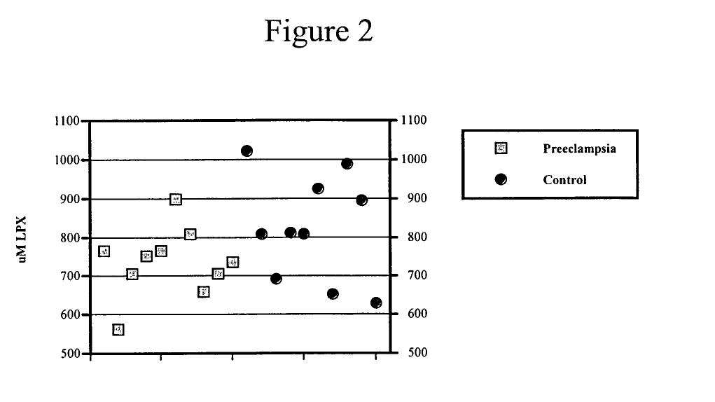

The concentrations of LPX in each sample were determined by subtracting the “background GPX” well G3P level from the G3P level detected in the “LPX+GPX” well of the samples. The concentrations in μM of GPX in each of the samples are presented in Table 1 and FIG. 1. The concentrations in μM of LPX in each of the samples are presented in Table 1 and FIG. 2. Preeclamptic blood samples were drawn from patients who were diagnosed with preeclampsia one week later. Normal blood samples, however, were drawn from patients who were not diagnosed with preeclampsia.

TABLE 1

| SAMPLE | DIAGNOSIS | GPX | LPX |
|---|---|---|---|
| 1 | Preeclampsia | 42 | 766 |
| 2 | Preeclampsia | 51 | 562 |
| 3 | Preeclampsia | 45 | 706 |
| 4 | Preeclampsia | 20 | 753 |
| 5 | Preeclampsia | 90 | 767 |
| 6 | Preeclampsia | 41 | 898 |
| 7 | Preeclampsia | 55 | 808 |
| 8 | Preeclampsia | 39 | 660 |
| 9 | Preeclampsia | 40 | 707 |
| 10 | Preeclampsia | 41 | 737 |
| 11 | Control | 113 | 1024 |
| 12 | Control | 107 | 809 |
| 13 | Control | 70 | 693 |
| 14 | Control | 71 | 813 |
| 15 | Control | 76 | 809 |
| 16 | Control | 101 | 925 |
| 17 | Control | 921 | 654 |
| 18 | Control | 188 | 989 |
| 19 | Control | 50 | 895 |
| 20 | Control | 35 | 628 |

Control Sample 17 was clearly aberrant, and so was discarded from mean and standard deviation calculations by accepted statistical methodology. As noted in the above cited co-pending applications, such abnormally high levels of glycerophosphatidyl compounds are sometimes observed in patients with disease states unrelated to preeclamptic pregnancy. As shown in Table 1, lower than normal levels of LPX were found generally in patients later developing preeclampsia. The average concentration of LPX from preeclampic samples measured 736.2±88.9 μM, whereas the average concentration of LPX in normal samples measured 842.7±130.1 μM. Concentrations of LPX in preeclamptic samples on average were 106 μM lower than concentrations in normal samples. Thus, the average concentration of LPX in the plasma of patients later developing preeclampsia was significantly lower than the average concentration of LPX in the plasma of healthy patients. Also shown in Table 1, lower than normal levels of GPX were found generally in patients later developing preeclampsia. The average concentration of GPX from preeclamptic samples measured 46.3±17.8 μM, whereas the average concentration of GPX in normal samples measured 90.2±44.8 μM. Concentrations of GPX in preeclamptic samples on average were 44 μM lower than concentrations in normal samples. Thus, the average concentration of GPX in the plasma of patients later developing preeclampsia was significantly lower than the average concentration of GPX in the plasma of healthy patients.

### Example 2

### Enzyme Cycling Assay of Plasma Specimen Levels of Lysophosphatidylcholine (LPC) and Glycerophosphatidylcholine (GPC) as Measured by Levels of Choline for the Detection of Preeclampsia

Reagents

Lysophospholipase (LYPL) was purchased from Asahi Chemical Industry, Tokyo, Japan. Glycerophosphorylcholine phosphodiesterase (GPC-PDE), choline oxidase, and 4-aminoantipyrine (AAP) were purchased from Sigma Chemical Co., St. Louis, Mo. Peroxidase was purchased from Boerhinger Mannheim, Indianapolis, Ind. 3,5 Dichloro-2-hydroxybenzenesulfonic acid sodium salt (HDCBS) was purchased from Biosynth AG, Naperville, Ill. All lipid and glycerophosphatidyl standards were purchased from Avanti Polar Lipids, Alabaster, Ala. or Sigma Chemical Co.

Sample Collection and Processing

Plasma was processed from blood collected as described in Example 1.

Enzymatic Assay

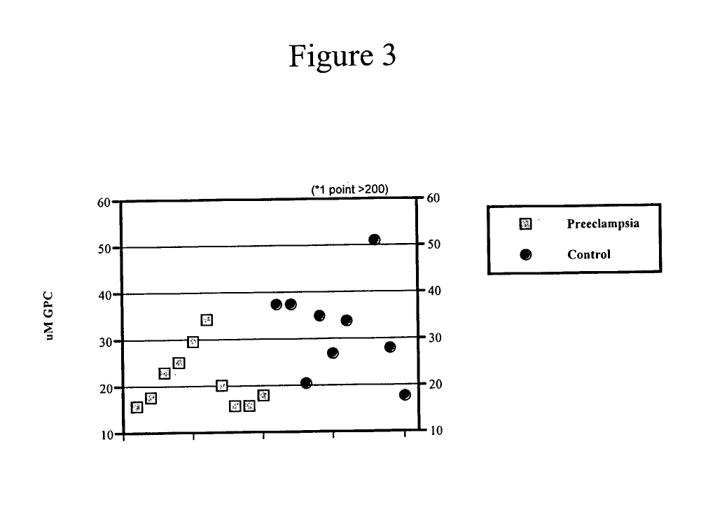

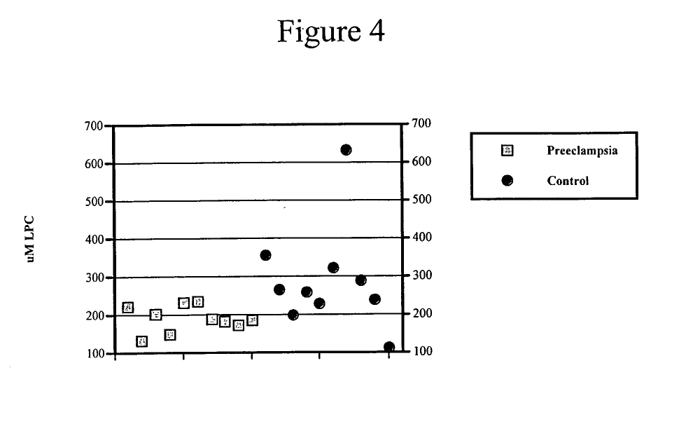

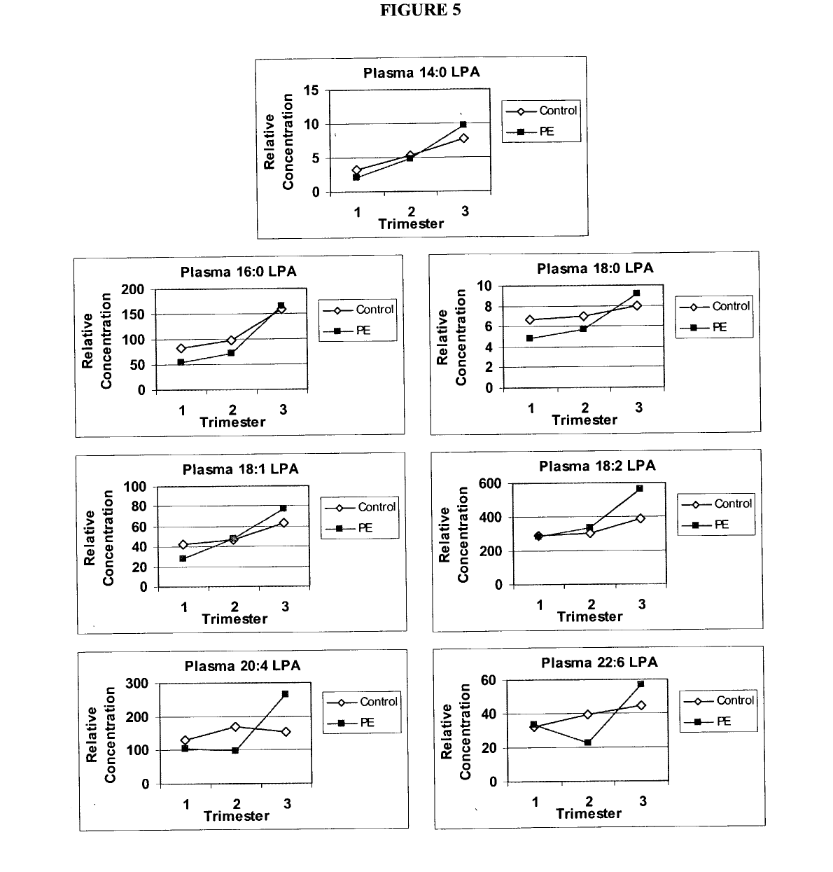

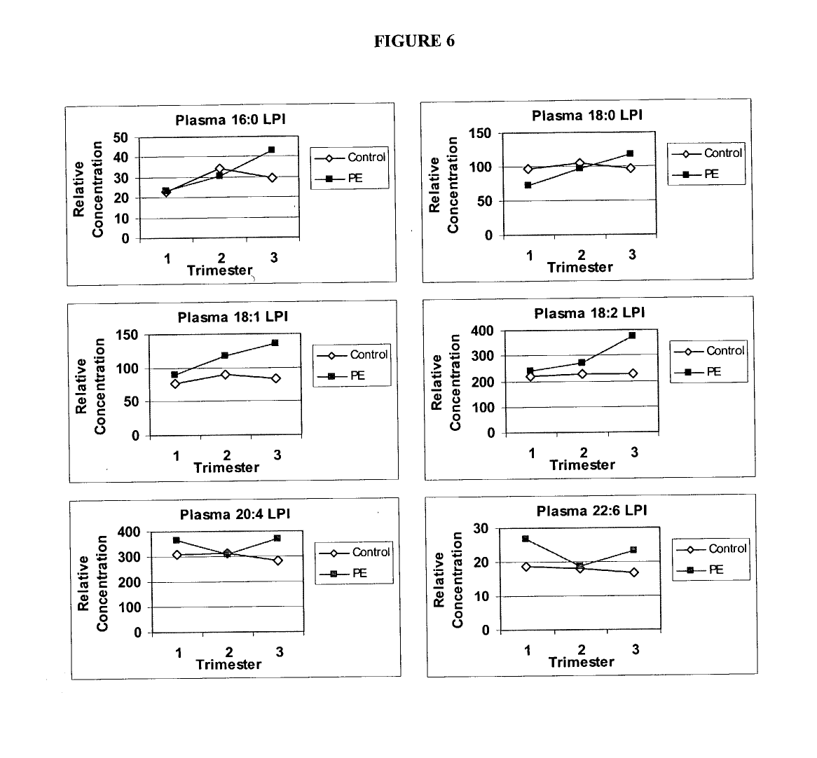

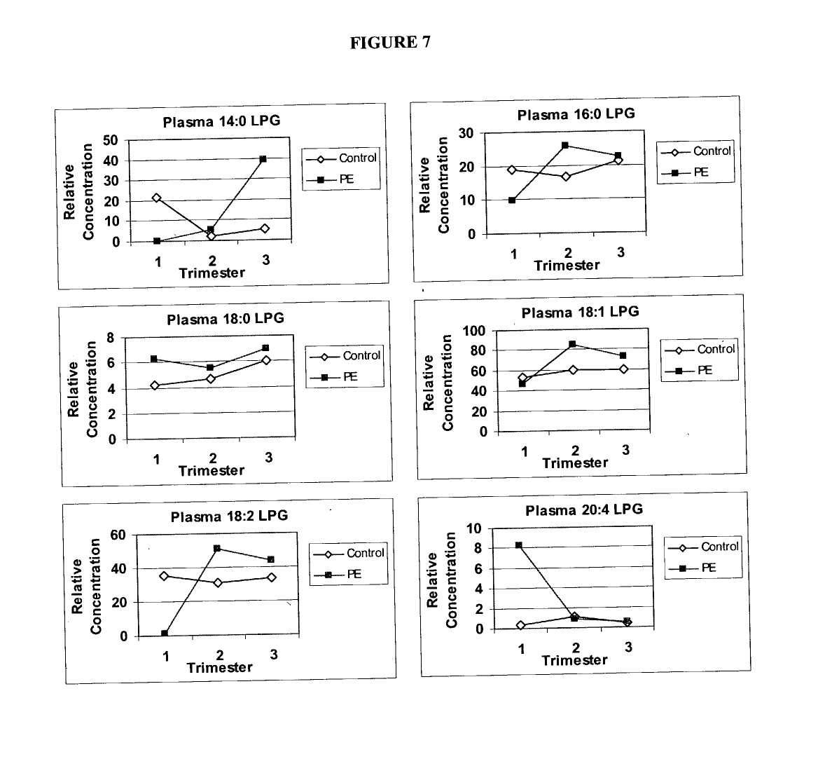

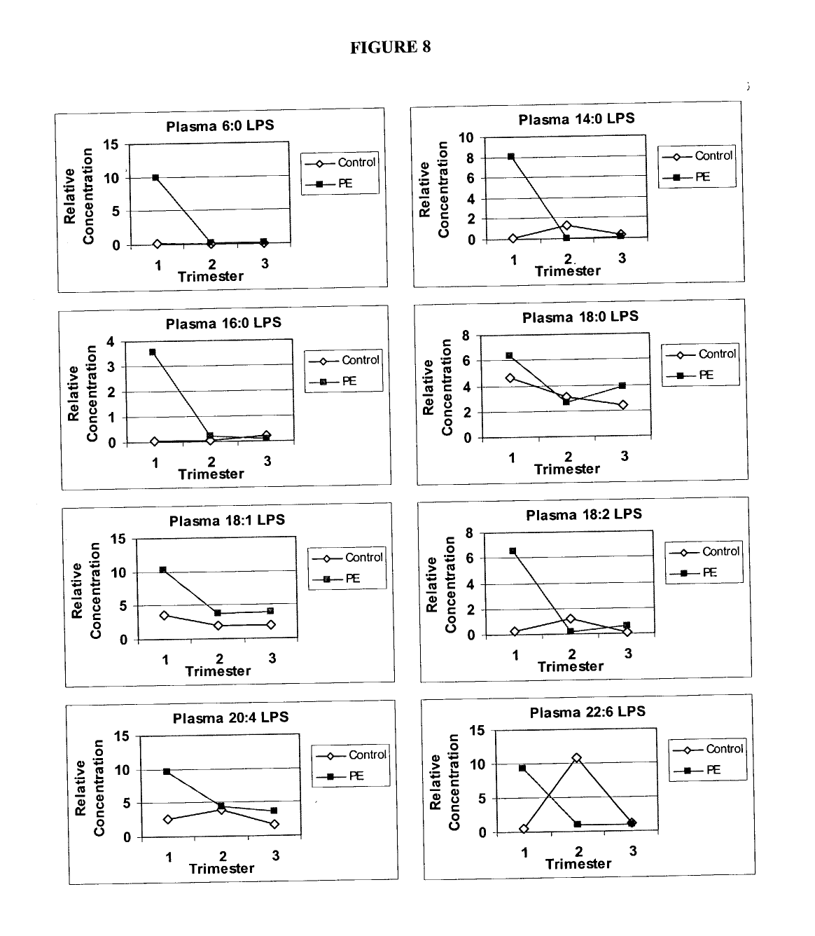

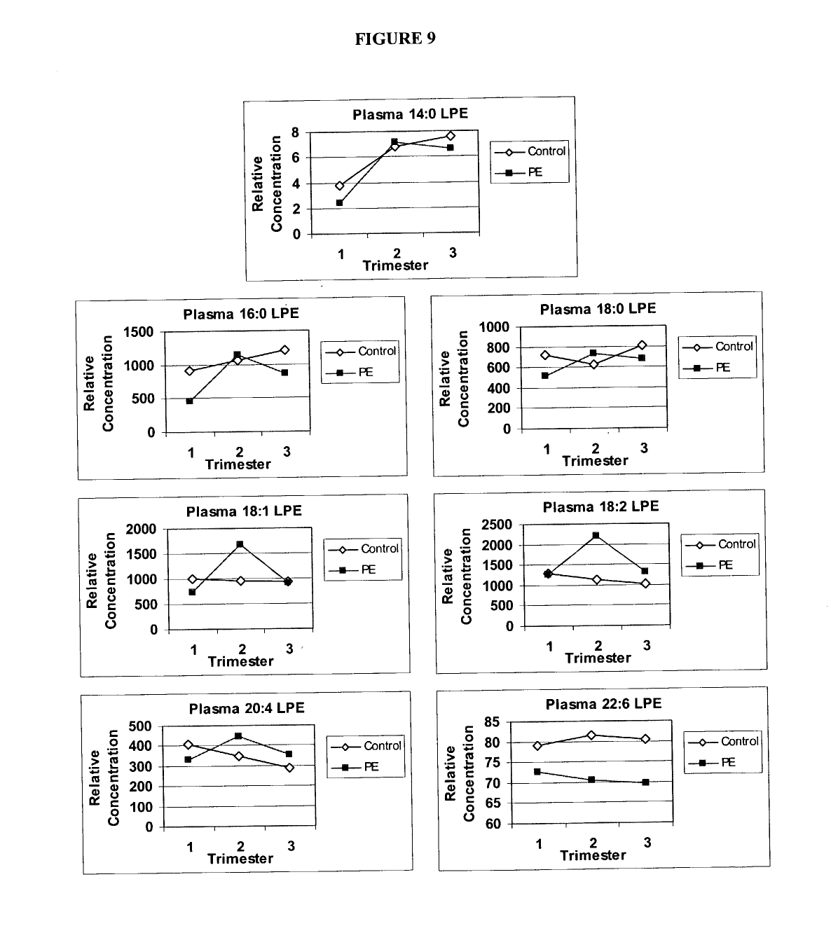

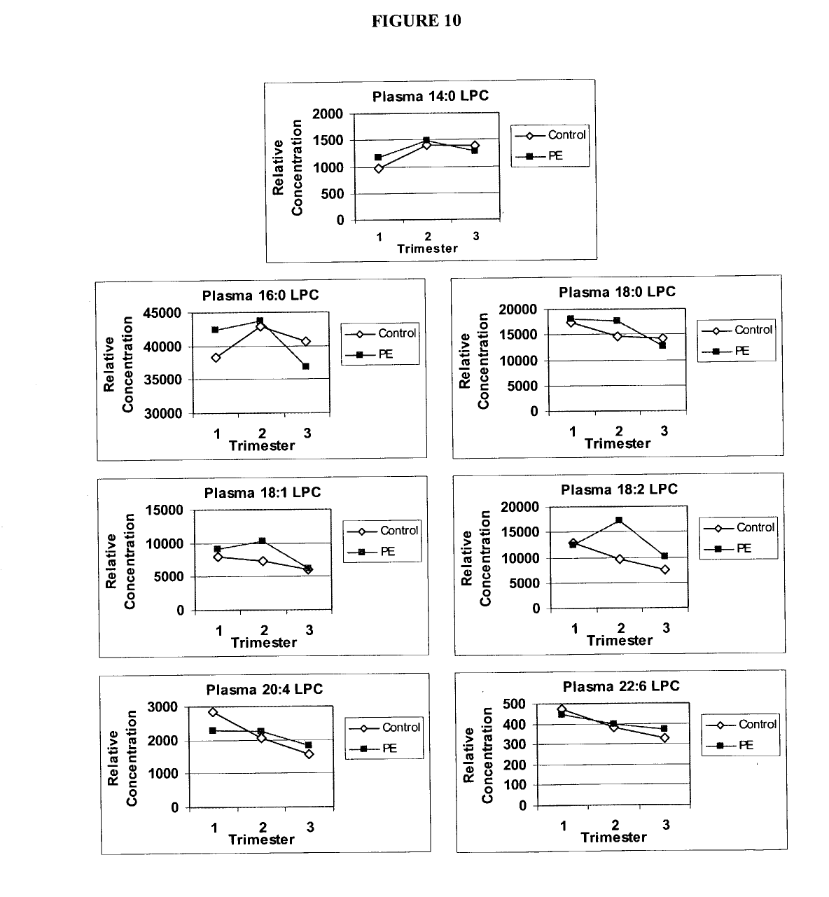

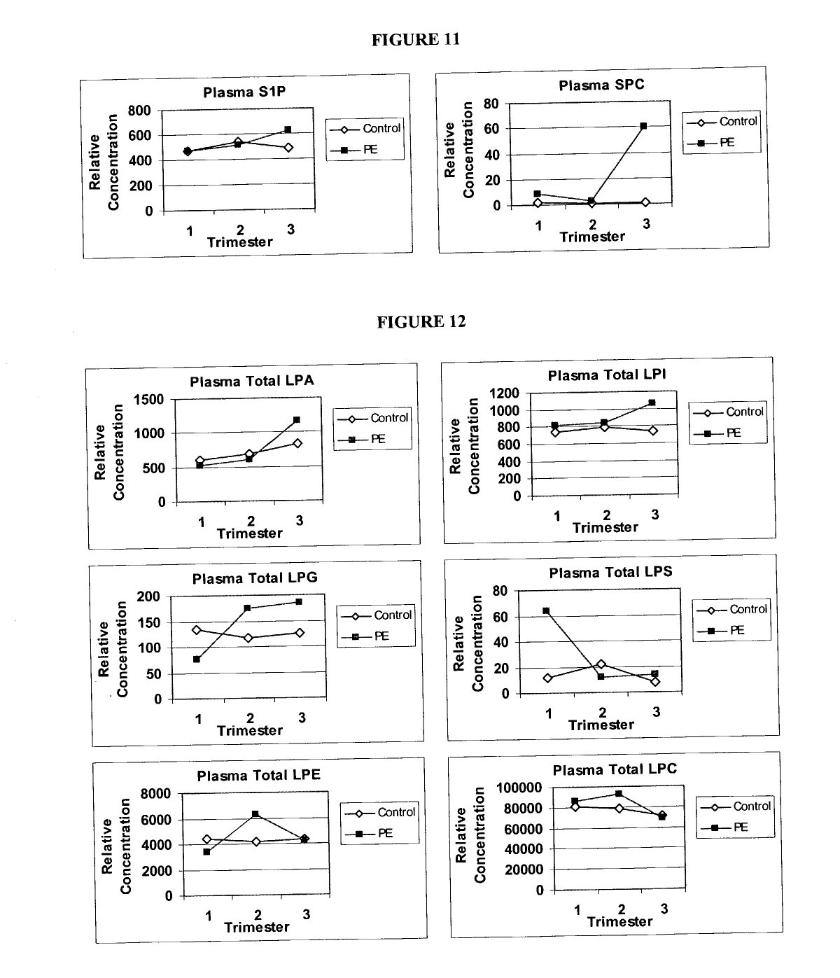

Using a 96 well microtiter plate, 5 μl of the sample were aliquotted into pairs of wells. To one well of each pair, the “LPC+GPC” well, 100 μl of LYPL (0.05 Units)/GPC-PDE (0.125 Units) were added. 100 μl of GPC-PDE (0.0125 Units) were added to the other “background GPC” well. The wells were incubated at 37° C. for 15 minutes. Glycerophosphorylcholine was produced as an intermediate by LYPL digestion of LPC. C3P and choline were then liberated from glycerophosphorylcholine using GPD-PDE. The plate was then blanked A505 in the ELISA reader, and 50 μl choline detection mix (0.15 Units choline oxidase, 0.5 Units peroxidase, 0.03% AAP, 0.125% HDCBS, 100 mM Tris pH 8.0) were added and incubated at 37° C. for 15 minutes. The plate was then read at A₅₀₅. The concentrations of LPC in each sample were determined by subtracting the “background” glycerphosphatidylcholine and choline levels from the choline level in the cleaved portions of the samples. TABLE 2 and FIG. 3 illustrate the results of the assay for GPC. TABLE 2 and FIG. 4 illustrate the results of the assay for LPC.

TABLE 2

| SAMPLE | DIAGNOSIS | GPC | LPC |
|---|---|---|---|
| 1 | Preeclampsia | 15.85 | 224 |
| 2 | Preeclampsia | 17.83 | 134 |
| 3 | Preeclampsia | 23.24 | 203 |
| 4 | Preeclampsia | 25.27 | 151 |
| 5 | Preeclampsia | 29.91 | 232 |
| 6 | Preeclampsia | 34.55 | 235 |
| 7 | Preeclampsia | 20.26 | 190 |
| 8 | Preeclampsia | 15.96 | 182 |
| 9 | Preeclampsia | 15.85 | 172 |
| 10 | Preeclampsia | 18.05 | 185 |
| 11 | Control | 37.46 | 355 |
| 12 | Control | 37.55 | 267 |
| 13 | Control | 20.59 | 198 |
| 14 | Control | 35.00 | 261 |
| 15 | Control | 27.09 | 231 |
| 16 | Control | 33.91 | 323 |
| 17 | Control | 208.59 | 632 |
| 18 | Control | 51.30 | 290 |
| 19 | Control | 28.27 | 238 |
| 20 | Control | 17.94 | 113 |

Control Sample 17 was clearly aberrant, and so was discarded from mean and standard deviation calculations by accepted statistical methodology. As noted in the above cited co-pending applications, such abnormally high levels of glycerophosphatidyl compounds are sometimes observed in patients with disease states unrelated to preeclamptic pregnancy. As seen in Table 2, lower than normal levels of LPC were generally found in patients later developing preeclampsia. Concentrations of LPC in patients with preeclampsia averaged 190.8±33.7 μM, whereas concentrations of LPC in normal patients averaged 252.9±71.1 μM. Thus, the average concentration of LPC in the plasma of patients diagnosed as having preeclampsia was significantly lower, by 62 μM, than the average concentration of LPC in the plasma of healthy patients. Also seen in Table 2, lower than normal levels of GPC were generally found in patients later developing preeclampsia. Concentrations of GPC in patients with preeclampsia averaged 21.7±6.5 μM, whereas concentrations of GPC in normal patients averaged 32.1±10.1 μM. Thus, the average concentration of GPC in the plasma of patients diagnosed as having preeclampsia was significantly lower, by 10 μM, than the average concentration of GPC in the plasma of healthy patients.

### Example 3

### Liquid Chromatography—Mass Spectrometry Analysis of Bioactive Lipids in Plasma and Serum Reagents

Methanol is purchased from Fisher Scientific. All lipid standards are purchased from Avanti Polar Lipids, Alabaster, Ala. or Sigma Chemical Co. LC-MS-MS triple quadrapole instrument is a Quattro Ultima (MicroMass, Beverly, Mass.). Tomtec model 320 liquid handler is purchased from Tomtec (Hamden, Conn). C18 columns are purchased from Keystone Scientific (Bellefonte, Pa.).

Sample Preparation

Plasma and serum samples are diluted in methanol in preparation for analysis on the LC-MS. Samples are diluted 1:5 by combining 75 μl of each sample with 300 μl methanol. Samples are then filtered into a collection plate to remove precipitated proteins. When possible, samples and methanol are pipetted using a Tomtec automated liquid handler. Alternatively, samples are extracted using methanol:chloroform (2:1), dried under nitrogen gas, and resuspended in methanol before being analyzed. Alternatively, samples are purified using solid phase extraction (SPE) such as reverse phase or anion exchange resins before being analyzed.

Liquid Chromatography-Mass Spectrometry

Sample separation and delivery to the mass spectrometer are performed using a Waters Alliance 2790 HPLC system (Waters, Milford, Mass.) with a C18 column (BetaBasic C18, 20×2 mm i.d., 5 μm particle size) obtained from Keystone Scientific (Bellefonte, Pa.). A gradient mobile phase is applied with solvent A containing methanol and solvent B containing water. In some cases, additives are included in solvent A and/or B to improve chromatography, such as phosphate buffer, formic acid, and ammonium acetate. Typically, chromatograms are about 10 minutes long with the following gradient conditions: Initial, 60% Solvent A, 40% Solvent B; 2 min: 60% Solvent A, 40% Solvent B; 8 min: 95% Solvent A, 5% Solvent B; 10 min: 95% Solvent A, 5% Solvent B. The flow rate is maintained at 0.2 ml/min.

Bioactive lipids of interest are quantified using the Quattro Ultima Triple Quadrapole mass spectrometer equipped with an ESI source. Instrument settings and tuning parameters are optimized for each lipid using standard lipid reagents as controls. Using multiple reaction monitoring (MRM) mode for maximum sensitivity, specific parent-daughter pairs are measured for each lipid of interest. Specific lipids elute from the LC column and are introduced into the mass spectrometer at different times during the analysis. Using this analysis, individual lipids or multiple lipids may be quantified during a single run.

Numerical values of specific lipid concentrations are obtained from standard curves constructed from known lipid amounts. A standard curve is analyzed for each separate lipid or class of lipid. In some cases, a known amount of an internal standard consisting of C¹³-labelled lipid is included in each sample to correct for sample-to-sample variation. Alternatively, lipids that contain fatty acid chains that do not naturally exist in these samples, such as 19:0 fatty acid, may be used as internal standards.

There will be various modifications, improvements, and applications of the disclosed invention that will be apparent to those of skill in the art, and the present application encompasses such embodiments to the extent allowed by law. Although the present invention has been described in the context of certain preferred embodiments, the full scope of the invention is not so limited, but is in accord with the scope of the following claims.

I claim:

1. A method for detecting preeclampsia by measuring bioactive lipids comprising:
obtaining a sample from a pregnant patient;
assaying the sample to determine the concentration of at least one bioactive lipid in the sample, wherein the lipid is selected from the group consisting of a glycerophosphatidyl compound, a lysophospholipid and a sphingolipid;
comparing the level of bioactive lipid in the sample to a control value or a prior sample from the pregnant patient; and
correlating the concentration in the sample to preeclampsia.

2. The method of claim 1, wherein the bioactive lipid is a lysophospholipid selected from the group consisting of LPA, LPI, LPG, LPS, LPC, and LPE.

3. The method of claim 1, wherein the bioactive lipid is a sphingolipid selected from the group consisting of S-1-P, SPC and sphingomyellin.

4. The method of claim 1, wherein the bioactive lipid is a glycerophospatidyl compound selected from the group consisting of GPA, GPI, GPG, GPS, GPC and GPE.

5. The method of claim 1, wherein the step of assaying the sample comprises measuring the concentration of a molecular species of the lysophospholipid.

6. The method of claim a, wherein the step of assaying the sample comprises measuring the concentration of the lipid and measuring the concentration of molecular species of the lipid and expressing the concentrations as a ratio.

7. The method of claim 1, wherein the specimen is serum.

8. The method of claim 1, wherein the specimen is plasma.

9. The method of claim 1, wherein the specimen is urine.

10. The method of claim 1, wherein the assaying step is repeated over time with a plurality of samples from the pregnant patient to monitor the condition.
